(19) United States
(12) Patent Application Publication
(10) Pub. No.: US 2020/0179296 A1
(43) Pub. Date: Jun. 11, 2020

(54) Accelerated Clearance of Biologic Therapeutics Using Click-Antidotes

(71) Applicant: The Regents of the University of Colorado, a body corporate, Denver, CO (US)

(72) Inventor: Dmitri Simberg, Centennial, CO (US)

(21) Appl. No.: 16/703,795

(22) Filed: Dec. 4, 2019

Related U.S. Application Data
(60) Provisional application No. 62/775,324, filed on Dec. 4, 2018.

(51) Int. Cl. A61K 9/51; A61K 47/64; A61K 9/107; A61K 31/495; A61K 31/015

(52) U.S. Cl. CPC A61K 9/5161; A61K 47/643; A61K 31/015; A61K 31/495; A61K 9/1075

(57) ABSTRACT

Click chemistry groups attached to both nanoparticles and antibodies to reduce antibody concentration in the body. The nanoparticles with attached click chemistry groups react with the antibody/biologic attached with a corresponding click chemistry group to sequester it in the liver and spleen, effectively and rapidly decreasing the concentration of free antibody/biologic.

### CROSS REFERENCE TO RELATED APPLICATION

This application claims the benefit of U.S. Provisional Patent Application Ser. No. 62/775,324, filed Dec. 4, 2018, which is incorporated herein by reference.

### STATEMENT OF FEDERALLY SPONSORED RESEARCH

This invention was made with government support under grant number EB022040 and CA194058 awarded by the National Institutes of Health. The government has certain rights in the invention.

### TECHNICAL FIELD

This invention relates generally to the field of nanoparticles, and in particular, to nanoparticles modified to include click chemistry to be used as an antibody antidote.

### BACKGROUND

Long blood half-life is one of the advantages of antibodies over small molecule drugs. At the same time, prolonged half-life is a problem for imaging applications or in case of antibody-induced toxicities. The renaissance of monoclonal antibodies has revolutionized medicine and the pharmaceutical industry. There are hundreds of clinically approved antibody drugs on the market and several hundred at different stages of clinical testing or approval. The majority of antibodies are designed for therapy, but some antibodies are also being tested for imaging, for example near infrared dye-labeled anti-epidermal growth factor (cetuximab) and anti-vascular endothelial growth factors (bevacizumab) for perioperative imaging. Unlike small molecule drugs and other biologics, antibodies possess intrinsically long half-lives that can be further enhanced through engineering. Among the factors affecting antibody longevity are affinity for FcRn (neonatal Fc receptor) that enables recycling after the internalization, overall charge, presence of sugar moieties, and molecular weight. There is a substantial need for antidotes that can quickly clear antibodies from systemic circulation and peripheral tissues.

The need for accelerated clearance of monoclonal antibodies was recognized many years ago when radionuclide-labeled antibodies were first tested in imaging applications, primarily in cancer. The wide body distribution and long circulation can lead to unnecessary exposure to radiation, prompting the development of a pretargeting approach, wherein non-radioactive antibody tagged with streptavidin was injected first, followed by injection of a biotinylated radioactive molecule. This pretargeting concept resulted in a much better signal-to-background ratio and image quality. At the same time, the long circulating properties of targeting antibodies were still a problem as one had to wait weeks until the antibody was sufficiently cleared for the imaging procedure to take place. Therefore, clearing strategies were developed that used, for example, neutralizing antibodies, galactosylated biotin albumin, biotin-albumin, avidin, complementary oligonucleotides, or extracorporeal affinity tags, in order to quickly eliminate the antibodies from the systemic circulation. Whereas some of these approaches, mostly biotin streptavidin pair, have been tested in nuclear imaging and therapy in patients, there is still a substantial risk of immunogenicity, as well as suboptimal clearing efficiency. More recently, several interesting clearing approaches to block FcRn recycling by in vivo PEGylation of the Fc portion or by anti-FcRn antibody, have been reported. However, these approaches still retain IgG in tissues, require introduction of non-natural amino acids into the antibody sequence or interfere with metabolism of natural immunoglobulins.

With the expanded range of antibody applications in clinical use, there is a substantial unmet need in antibody antidotes. For example, immune checkpoint inhibitors (anti-CTLA-4 and anti-PD-1) cause serious dermatologic and neurological toxicities, whereas anti-EGFR antibody causes severe skin toxicity, and there are no effective strategies to eliminate these drugs from the body once the adverse effects appear. In addition, antibodies for infrared perioperative imaging are directly labeled and may take days until cleared from circulation. Bioorthogonal click chemistry, in particular copper-free Diels-Alder additions involving strained trans-cyclooctene (TCO) and methyltetrazine (MTZ) has been proven to be versatile due to very fast second order reaction rates resulting in the formation of a stable covalent bond. The efficient pretargeting and in vivo cell surface modification using TCO-MTZ pair have been previously demonstrated.

### SUMMARY

This application describes a set of clinically relevant nano-sized drug carriers with different circulation properties that have been created as click chemistry antidotes. Click-modified nanoparticles should interact with click-modified antibodies in vivo and trigger elimination from the blood stream, due to the general propensity of nanomedicines to be cleared by the liver and spleen. These results demonstrate that the clearance is dictated by the elimination half-life of the antidote as well as the tissue/blood distribution of the antibody. This invention provides a cost-effective, clinically viable antibody clearing nano-antidotes.

Engineered nanoparticles exhibit intrinsic affinity for clearance organs (mainly liver and spleen). Trans-cyclooctene (TCO) and methyltetrazine (MTZ) are versatile copper-free click chemistry components that are extensively used for in vivo biorthogonal couplings. To demonstrate the ability of nanoparticles to eliminate antibodies, a set of click-modified, clinically relevant antidotes based on several classes of drug carriers: phospholipid-PEG micelles, bovine serum albumin (BSA), and cross-linked dextran iron oxide nanoparticles (CLIO), were prepared. Mice were injected with IRDye 800CW-labeled, click modified IgG followed by a click-modified antidote or PBS (control), and the levels of the IgG were monitored up to 72 h post injection. Long-circulating lipid micelles produced a spike in IgG levels at 1 h, decreased IgG levels at 24 h, and did not decrease the area under the curve (AUC) and IgG accumulation in main organs. Long-circulating BSA decreased IgG levels at 1 h and at 24 h, decreased the AUC, but did not significantly decrease organ accumulation. Long-circulating CLIO nanoworms increased IgG levels at 1 h, decreased IgG levels at 24 h, did not decrease the AUC, and did not decrease the organ accumulation. On the other hand, short-circulating CLIO nanoparticles decreased IgG levels at 1 h and 24 h, significantly decreased the AUC and accumulation in the main organs. Multiple doses of CLIO and BSA were not able to completely eliminate the antibody from blood, despite the click reactivity of the residual IgG, likely due to exchange of IgG between blood and tissue compartments. Pharmacokinetic modeling suggests that short antidote half-life and fast click reaction rate should result in higher IgG depletion efficiency. In conclusion, short-circulating click-modified nanocarriers are the most effective antidotes for elimination of antibodies from blood.

Further, a method for reducing click-modified antibodies from a subject using the disclosed click-modified nanoparticles is provided. The compounds of this disclosure having antibody-clearing properties are useful for reducing the toxic and adverse side effects of said antibodies. In particular, the compounds of this disclosure may be used as an antidote to adverse events developed following administration of the antibodies.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the figures.

### BRIEF DESCRIPTION OF FIGURES

FIGS. 1A-1F show the antidotes used in the study. FIG. 1A shows the types of nanocarriers used to prepare antidotes. The nanocarriers were modified with click groups (DSPE-PEG3400 with MTZ, BSA with TCO, CLIO NW and CLIO with TCO) as described in Methods. FIG. 1B shows TEM images of iron oxide cores of CLIO NWs and CLIO. Crosslinked dextran shell is not visible. Size bar=50 nm. FIGS. 1C-1F show the elimination profile of the antidotes and organ distribution (NIR images). Labels are as follows: k-kidney, s-spleen, Iv-liver, i-intestine, lu-lung, h-heart). DSPE-PEG-MTZ, BSA-TCO and CLIO NW-TCO are long circulating particles, whereas CLIO-TCO are short circulating particles. Each time point shows mean and SD. N=3 mice per group. FIG. 1C shows the elimination profile for DSPE-PEG-MTZ. FIG. 1D shows the elimination profile for BSA-TCO. FIG. 1E shows the elimination profile for CLIO NW-TCO. FIG. 1F shows the elimination profile for CLIO-TCO.

FIGS. 2A-2E show the depletion efficiency of IgG in vivo by the antidotes: FIG. 2A shows IgG labeled with IRDye 800CW and either TCO or MTZ and injected into female BALB/c mice (25 μg/mouse), followed by antidote or PBS (control). Blood IgG levels were measured at different time points, up to 72 h post injection. FIG. 2B shows DSPE-PEG-MTZ. Insert shows early time points for DSPE-PEG-MTZ; FIG. 2C shows BSA-TCO; FIG. 2D shows CLIO NW-TCO; and FIG. 2E shows CLIO-TCO. Black line is “IgG only”, green line is “IgG+antidote”. Time of IgG injection is “0” on the X-axis. The antidotes were injected at different times after IgG injection as marked by red arrows. First blood draw was at 1 h post-IgG injection (100%). The IgG only (control) group was included in every experiment. Each time point shows mean and SD. Two tailed t-test, n=3 mice per group.

FIGS. 3A-3D show the organ distribution in control and antidote-injected groups: FIG. 3A shows DSPE-PEGMTZ; FIG. 3B shows BSA-TCO; FIG. 3C shows CLIO NW-TCO; and FIG. 3D shows CLIO-TCO. Graphs show mean gray value of fluorescence scans, images show colored images in 800 nm channel. Organs were scanned in 24 well plates; each organ's image corresponds to the bar graph below; representative images are shown. The signal represents both circulating IgG and organ-deposited IgG. Brightness and contrast were adjusted to the same extent in control and treated groups of the same experiment. Due to scanner settings, antibody batch and time when the experiment was terminated, the grey values show differences between experiments. 2-way ANOVA test with multiple comparisons, n=3 mice per group. Means and SD of experimental replicates are shown

FIG. 4 shows histological images of liver of mice injected with IgG-MTZ or with IgG-MTZ followed by CLIO-TCO. In the “IgG+CLIO-TCO” group, the antibody and the nanoparticles mostly co-localize in Kupffer cells (light gray) with some endothelial staining, whereas in the “IgG only” group there is a diffuse fluorescence of IgG, with some of the antibody accumulating in hepatocytes and sinusoidal endothelium. The experiment was done in 2 mice per group; representative images are shown.

FIGS. 5A-5C show multiple injections of antidotes do not improve the efficiency of depletion. IgG-MTZ was injected in mice and then followed with 3 injections of the antidote. FIG. 5A shows injections with CLIO-TCO. FIG. 5B shows injection with BSA-TCO. Arrows show injection of antidote, black lines are IgG only group, gray lines are antidote group. First blood draw was at 1 h post-injection of IgG (100%). None of the antidotes were able to deplete all IgG after multiple injections. Each time point shows mean and SD. N=3 mice per group. FIG. 5C shows the AUC24 h summary for all the experiments (FIGS. 2B-2E and FIGS. 5A-5B) shows no difference in depletion efficiency between 1 and 3 injections of CLIO-TCO, and no difference between 2 and 3 injections of BSA-TCO. Note lack of depletion by DSPE-PEG-MTZ and increased AUC24 h by CLIO NW-TCO demonstrate that nanoparticles keep the antibody from elimination (2-sided t-test, n=3 mice per group).

FIG. 6 shows remaining IgG in blood is click-reactive. Mice were injected with IRDye 800CW-IgG-MTZ (control) or with IRDye 800CW-IgG-MTZ followed by CLIO-TCO as described in FIG. 2E, and blood samples were collected at 4.5 h and 24 h post injection. CLIO-TCO was added to blood samples ex vivo and pulled down with ultracentrifugation as described in Methods. Bar graphs and corresponding dots (3 mice) show supernatant NIR fluorescence. CLIO-TCO was able to completely pull down the remaining antibody, suggesting the click reactivity. Note the difference in blood IgG levels between control and CLIO-TCO injected mice at both time points. Means and SD of 3 mice are shown.

FIGS. 7A-7F show pharmacokinetic modeling of depletion efficiency: FIG. 7A is a schematic of the PK model. Upper model was used to fit IgG (control) data, and lower model was used to fit IgG+antidote data. Gray circles denote blood compartment, black circles denote tissue compartment. IgG data are blood data for control and antidote injected mice, respectively, k₁₂ and k₂₁ are exchange rates constants with the tissue compartment; kR is click reaction rate constant (2nd order); kₑₗ is IgG elimination rate constant; kA is antidote elimination rate and kA-IgG is antidote-IgG elimination rate constant. The fitted and model-adjusted values are in Table 2. FIG. 7B shows model-fitted (solid line) and actual (solid black symbols) blood IgG levels for CLIO-TCO (1 injection, FIG. 2E). FIG. 7C shows model-fitted (solid line) and actual (solid black symbols) blood IgG levels for CLIO-TCO (3 injections, FIG. 5A). FIG. 7D shows model-fitted (solid line) and actual (solid black symbols) blood IgG levels for CLIO NW-TCO. Note that the model accurately predicts spike in IgG concentration at early time points. FIG. 7E shows a simulation of the effect of click reaction rate constant on IgG elimination profile. Higher click reaction rate constant leads to better elimination efficiency. For comparison, the fitted data for CLIO-TCO are shown (gray line). FIG. 7F shows a simulation of an ideal hypothetical antidote (light gray line) that has a slow elimination rate constant but 100 times faster elimination rate constant once bound to IgG. For comparison, the fitted data for CLIO-TCO are shown (gray line).

FIG. 8 shows the scheme for synthesis of DSPE-PEG3400-MTZ.

FIG. 9 shows the elimination profile of IgG. IRDye800CW-labeled human polyclonal IgG (25 μg) was injected into 3 BALB/c female mice. Blood samples at different time points were lysed, blotted on a nitrocellulose membrane and scanned for 800 nm fluorescence using Li-COR Odyssey. The absolute levels of IgG in blood were measured from standard dilution of IgG in lysed mouse blood scanned on the same membrane. N=3 mice.

FIG. 10 shows the reactivity of CLIO-TCO and CLIO NW-TCO with IgG in mouse blood. IRDye800CW-IgG-MTZ was spiked into fresh heparinated mouse blood, and the nanoparticles were added to blood and incubated as described in the Detailed Description (15 μg IgG/90 μg Fe). Blood cells and particles were pelleted with ultracentrifuge and the fluorescence in the supernatant was measured. IRDye 800CW fluorescence is higher in blood than in PBS. The depletion efficiency was ˜70%. The ratio of NP/IgG was 2 times lower than used in vivo (25 μg IgG/290 μg Fe). The experiment was repeated 2 times.

FIGS. 11A and 11B show the reaction between CLIO-TCO and IRDye 800CW-IgG-MTZ in vitro. FIG. 11A shows that the reaction in vitro does not lead to the fluorescence quenching of IgG. FIG. 11B shows only CLIO-TCO but non unmodified CLIO reacts with IRDye 800CW-IgG-MTZ in presence of 90% FBS. Image shows a representative dot blot of supernatant after pelleting of nanoparticles. The experiment was repeated 2 times. Mean and SD of 3 technical replicates are shown.

FIG. 12 shows the elimination half-life of IgG-MTZ with or without CLIO-TCO. The antibody was indirectly detected in plasma dots using goat anti-human IRDye 680 (Li-COR Biosciences). N=2 mice per group.

FIG. 13 shoes the liver accumulation of IgG-MTZ with or without CLIO-TCO. The antibody was indirectly detected in tissue sections using goat anti-human IRDye 680 (Li-COR Biosciences). Representative images (2 mice per group) are shown.

FIG. 14 shows the hematological counts of mice injected with IgG-MTZ (mouse 1, 2) or IgG-MTZ followed by CLIO-TCO (mouse 3, 4). Blood was taken 4 h post injection of IgG-MTZ and analyzed with Element HT5 Veterinary Hematology Analyzer (HESKA, Loveland, Colo.). There were no abnormalities except borderline decrease in platelets in mouse 4.

FIG. 15 shows the H&E staining of organs of mice injected with IgG-MTZ (top panel) or IgG-MTZ and CLIO-NW-TCO (bottom panel). Representative images (2 mice per group) are shown.

FIGS. 16A and 16B show the elimination half-life of CLIO-TCO and CLIO-NW-TCO with or without conjugation to IgG-MTZ. FIG. 16A shows the elimination half-life of CLIO-NW-TCO with or without conjugation to IgG-MTZ. FIG. 16B shows the elimination half-life of CLIO-TCO with or without conjugation to IgG-MTZ. Nanoparticles labeled with IRDye 800CW were reacted with excess non-labeled IgG-MTZ and injected into female BALB/c mice (n=3/group) as described in main Methods. At the end of the experiments (48 h) organs were scanned with Li-COR Odyssey. Conjugation of IgG does not affect elimination and biodistribution of the antidotes. N=3 mice per group.

### DETAILED DESCRIPTION

This disclosure provides compounds combining copper-free click chemistry components with nanoparticle carriers that may be used to prevent, treat, or ameliorate adverse reactions or toxicities to antibodies in a subject by administering a therapeutically-effective amount of a compound of this disclosure that reduces the concentration of antibodies in the subject's system. Further, methods for reducing antibody concentration in a subject using the disclosed compounds is provided. These methods include administering a therapeutically effective amount of a disclosed compound to a subject to achieve a reduced concentration of a particular click-chemistry modified antibody.

The disclosed compounds can be used in combination with other compositions and procedures for the prevention and treatment of adverse reactions. For example, a subject may be treated conventionally with an antihistamine in combination with one or more of the click-modified nanoparticle compounds disclosed herein.

The disclosed compounds may be combined with pharmaceutically acceptable excipients, and optionally sustained-release matrices, such as biodegradable polymers, to form therapeutic compositions. Therefore, also disclosed are pharmaceutical compositions including one or more of any of the compounds of this disclosure and a pharmaceutically acceptable carrier.

These pharmaceutical compositions can be in the form of tablets, capsules, powders, granules, lozenges, liquid or gel preparations, such as oral, topical, or sterile parenteral solutions or suspensions (e.g., eye or ear drops, throat or nasal sprays, etc.), transdermal patches, and other forms known in the art.

Pharmaceutical compositions can be administered systemically or locally in any manner appropriate to the treatment of a given condition, including orally, parenterally, intrathecally, rectally, nasally, buccally, vaginally, topically, optically, by inhalation spray, or via an implanted reservoir. The term “parenterally” as used herein includes, but is not limited to subcutaneous, intravenous, intramuscular, intrasternal, intrasynovial, intrathecal, intrahepatic, intralesional, and intracranial administration, for example, by injection or infusion. For treatment of the central nervous system, the pharmaceutical compositions may readily penetrate the blood-brain barrier when peripherally or intraventricularly administered.

The pharmaceutical compositions can also be administered parenterally in a sterile aqueous or oleaginous medium. The composition can be dissolved or suspended in a non-toxic, parenterally-acceptable diluent or solvent, e.g., as a solution in 1,3-butanediol. Commonly used vehicles and solvents include water, physiological saline, Hank's solution, Ringer's solution, and sterile, fixed oils, including synthetic mono- or di-glycerides, etc. For topical application, the drug may be made up into a solution, suspension, cream, lotion, or ointment in a suitable aqueous or non-aqueous vehicle. Additives may also be included, for example buffers such as sodium metabisulphite or disodium edeate; preservatives such as bactericidal and fungicidal agents, including phenyl mercuric acetate or nitrate, benzalkonium chloride or chlorhexidine, and thickening agents, such as hypromellose.

The dosage unit involved depends, for example, on the condition treated, nature of the formulation, nature of the condition, embodiment of the claimed pharmaceutical compositions, mode of administration, and condition and weight of the patient. Dosage levels are typically sufficient to achieve a tissue concentration at the site of action that is at least the same as a concentration that has been shown to be active in vitro, in vivo, or in tissue culture. For example, a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day can be useful for treatment of a particular condition.

Materials: Iron salts were purchased from Sigma-Aldrich (St Louis, Mo., US). DSPEPEG3400-amine was from Laysan Bio. Bovine serum albumin was from Sigma (St. Louis, Mo., USA). Methyltetrazine (MTZ)-PEG4-NHS ester, Trans-Cyclooctene (TCO)-PEG4-NHS ester and MTZ-NHS ester were from Click Chemistry Tools (Scottsdale, Ariz., USA). IRDye 800CW-NHS ester was from Li-COR Biosciences (Lincoln, NB, USA). Purified human IgG was from Jackson ImmunoResearch (West Grove, Pa., USA). Zeba desalting columns (7 kDa and 40 kDa) were from Thermo Fisher. Cy7-NHS ester and Cy3-NHS ester were from Lumiprobe Inc. (Hunt Valley, Md., USA). Goat anti-human IRDye680 antibody was from Li-COR. Lipophilic carbocyanine dye DiOC18(7) (“DiR”) was from Thermo Fisher.

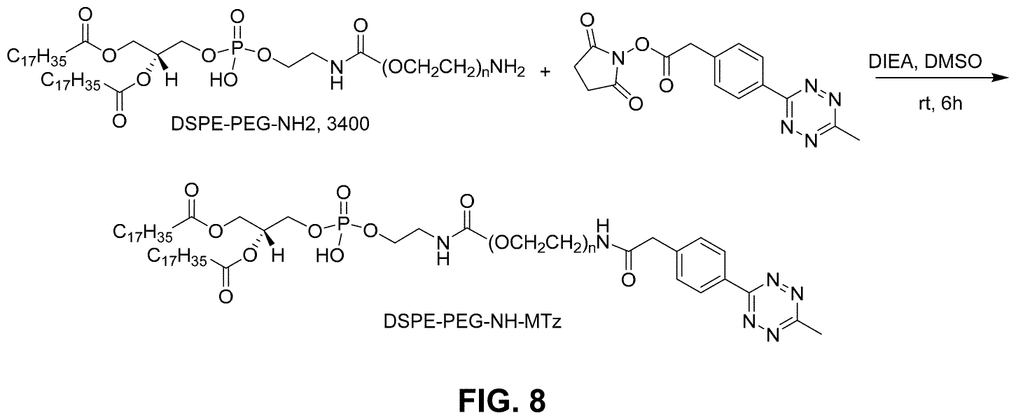

Synthesis of DSPE-PEG3400-MTZ: DSPE-PEG-MTZ was synthesized according to the scheme depicted in FIG. 8 by reacting methyltetrazine NHS ester (4.33 mg, 13.2 μmol) with DSPEPEG3400-NH2 (MW=3400, 30 mg, 8.8 μmol) and DIEA (3.42 mg, 26.47 μmol) in 1 mL anhydrous DMSO at room temperature for 6 h. The crude product was purified by C18 reverse phase HPLC and the product eluted with 95% Methanol/water, to obtain DSPEPEG-MTZ as a red solid (20.2 mg, 63% yield). 1H-NMR (400 MHz, CDCl3): δ 0.90 (m, 10H); 1.27 (m, 56H); 1.46-1.57 (m, 9H); 2.23-2.56 (m, 18H); 3.12 (s, 3H); 3.39-3.52 (m, 3H); 3.66 (m; 269H); 3.82 (s, 3H); 4.08 (s, 2H); 4.09-4.26 (m, 4H); 4.35 (m, 1H); 5.20 (m, 1H); 6.43 (m, 1H); 7.28 (d, J=8.40 Hz, 2H, Ar—H); 7.82 (s, 1H); 8.57 (d, J=8.36 Hz, 2H, Ar—H).

Synthesis of BSA-TCO and IRDye 800-BSA-TCO: Bovine serum albumin (10 mg/mL in 100 μL PBS) was combined with a 10-fold excess of TCO-PEG4-NHS ester (2 μL DMSO). The reaction was allowed to incubate at 4° C. for 24 h. Alternatively, BSA in PBS (100 μL, 5 mg/mL) was combined with a 10-fold excess of IRDye 800CW-NHS ester (in 2 μL DMSO) for 15 min before adding a 20-fold excess of TCO-PEG4-NHS ester (in 2 μL DMSO). All the reactions were purified using a 7,000 molecular weight cutoff Zeba spin column.

Synthesis of CLIO and CLIO NWs: CLIO NWs were synthesized from native 20 kDa dextran (Sigma) whereas CLIO were synthesized from reduced T-10 (10 k Da) dextran (Pharmacosmos). Dextran, Fe (III) chloride and Fe (II) chloride were mixed in DDW and titrated with ammonia using a modified Molday precipitation method. The ratio between dextran and iron salts determined the final size of the nanoparticles. Excess dextran was removed with ultrafiltration and the particles were crosslinked with epichlorohydrin as described. Excess epichlorohydrin was removed with ultrafiltration and particles were aminated by addition of ammonium hydroxide (CLIO NWs) or diaminohexane (CLIO) overnight at 4° C. Excess of amines was removed by ultrafiltration; particles were resuspended in sterile water, filtered through a 0.45 μm filter and stored at 4° C. Size and zeta potential were determined using Zetasizer Nano ZS (Malvern Instruments Ltd, Malvern, UK). The intensity-weighted distribution was used to report hydrodynamic diameter. For TEM analysis, the imaging of non-stained samples applied on carbon grid was performed using FEI Tecnai Spirit BioTwin electron microscope at 100 keV. The amino group content was determined by reaction with Cy7-NHS. Particles were reacted in PBS with excess dye and then precipitated with 99% ethanol. The amount of the reacted dye was determined by the difference in the supernatant fluorescence between the reaction tube and control tube (no particles). The number of amines per nanoparticle was determined to be ˜100,000 per CLIO and CLIO NW.

Synthesis of CLIO-TCO and IRDye 800CW-CLIO-TCO: Aminated CLIO in H2O (100 μL, 4 μM) was combined with a 100-fold excess of TCO-PEG4-NHS ester (in 2 μL DMSO). The reaction was allowed to incubate at 4° C. for 24 h and was purified with a 40 kDa molecular weight cut off Zebra spin column. Alternatively, aminated CLIO in H2O (100 μL, 4 μM) was combined with a 30-fold excess of IRDye 800CW-NHS ester (in 2 μL DMSO) for 15 min before adding a 100-fold excess of TCO-PEG4-NHS ester (in 2 μL DMSO). The reaction was allowed to incubate at 4° C. for 24 h and was purified using a 40 k Da cut off Zeba spin column.

Synthesis of CLIO NW-TCO and IRDye 800CW-CLIO NW-TCO: Aminated CLIO NW in H2O (100 μL, 500 nM) was combined with a 100-fold excess of TCO-PEG4-NHS ester (in 2 μL DMSO). The reaction was allowed to incubate at 4° C. for 24 h. Alternatively, aminated CLIO NW in H2O (100 μL, 500 nM) was combined with a 30 fold excess of IRDye 800CW NHS ester (in 2 μL DMSO) for 15 min before adding a 100 fold excess of TCO-PEG4-NHS ester (in 2 μL DMSO). The reaction was allowed to incubate at 4° C. for 24 h and was purified using a 40 kDa cutoff Zeba spin column.

Synthesis of IgG-MTZ, IRDye 800-IgG-MTZ and IRDye 800-TCO: Human polyclonal IgG in PBS (100 μL, 10 mg/mL) was combined with a 20-fold excess of MTZ-PEG4-NHS ester or TCO-PEG4-NHS (in 2 μL DMSO). The reaction was allowed to incubate at 4° C. for 24 h and was purified using a 7 kDa cut off Zebra spin column.

Alternatively, IgG in PBS (100 μL, 10 mg/mL) was combined with a 10-fold excess of IRDye 800CW-NHS ester (in 2 μL DMSO) for 15 min before adding a 20-fold excess of methyltetrazine-PEG4-NHS ester (in 2 μL DMSO). The reaction was allowed to incubate at 4° C. for 24 h and was purified using a 7 kDa cut off Zebra spin column. Conjugation efficiency was about 1-2 IRDye 800/IgG as determined by UV absorbance and the dye extinction coefficient of 160,000 M−1 cm−1.

In vitro reactivity of nanoparticles: To compare the efficiency of reaction of CLIO NWTCO and CLIO-TCO with IgG-MTZ in mouse blood, IRDye 800-IgG-MTZ was added to fresh heparinized mouse blood or 1% BSA/PBS at 15 μg/mL. CLIO-NW and CLIO-NW TCO were added next at 90 μg/mL. The reaction was mixed at 37° C. for 10 min and then centrifuged at 450,000 g in TLA-100.3 rotor (Beckman Optima ultracentrifuge) in order to pellet blood cells and nanoparticles. Supernatants were collected into a 384 well black/transparent plate and scanned for NIR fluorescence with Li-COR Odyssey at 800 nm. The fluorescence of supernatant without added particles (control) was set as 100%.

For measurement of quenching of IRDye 800CW fluorescence, CLIO-TCO were mixed at different weight ratios with IRDye 800CW-IgG-MTZ or IRDye 800CW-IgG (control), incubated for 15 min and the fluorescence was scanned at 800 nm with Li-COR Odyssey. IgG fluorescence without added nanoparticles was set as 100%.

To test specificity of reaction of CLIO-TCO, 1.5 μg of IRDye 800CW-IgG-MTZ was added to 100 μL FBS. Aminated CLIO or CLIO-TCO were added in triplicates to FBS (15 μg Fe/sample), incubated for 1.5 h at room temperature, and pelleted at 450,000 g for 15 min by ultracentrifugation as described above. 2 μL of supernatant was blotted for each of the samples on nitrocellulose membrane and scanned at 800 nm.

In order to test reactivity of the residual circulating antibody that was not depleted by CLIO-TCO, mice (injected with IgG only (control) or IgG followed by CLIO-TCO) were bled at 4.5 h and 24 h post IgG injection. After the blood samples (˜50 μL) were lysed, the lysates were split into two groups. One group had 200 μg of CLIO-TCO added to it, and the other group had PBS. After 3 h of incubation both groups were spun down at 450,000 g and the lysates were blotted on a nitrocellulose membrane and scanned by Li-COR at 800 nm.

Mouse experiments: The University of Colorado Institutional Animal Care and Use Committee (IACUC) approved all animal experiments. Mice were treated according to regulations provided by the Office of Laboratory Animal Resources at the UC Denver.

Female BALB/c mice 6-8 weeks old were used for the experiments and bred in house. In order to determine circulation half-life of NIR-labeled antibodies and antidotes, 2 μL blood were collected at different time points through retroorbital vein or mandibular vein and were applied in duplicates on a 0.22 μm nitrocellulose membrane and scanned at 800 nm using Li-COR Odyssey. The spot integrated density of a 16-bit TIFF image was measured with ImageJ and plotted as a function of time. The fluorescence of dots at initial time point was used as 100%. For organ distribution, mice were euthanized at 48-72 h post injection and the organs were placed in wells on a 12-well plate. Organs were scanned with Li-COR Odyssey at 800 nm. Mean fluorescence was determined from 16-bit images using ImageJ software by subtracting the background, drawing a ROI around the organs, and using a measure function to determine mean gray value.

In order to compare elimination half-lives of the antidotes alone and conjugated to IgG, IRDye 800CW-CLIO NW-TCO and IRDye 800CW-CLIO-TCO were treated with excess of IgG-MTZ in 10% FBS in PBS overnight (4° C.). The antidotes with or without IgG (30 μg Fe) were injected in 3 mice per group.

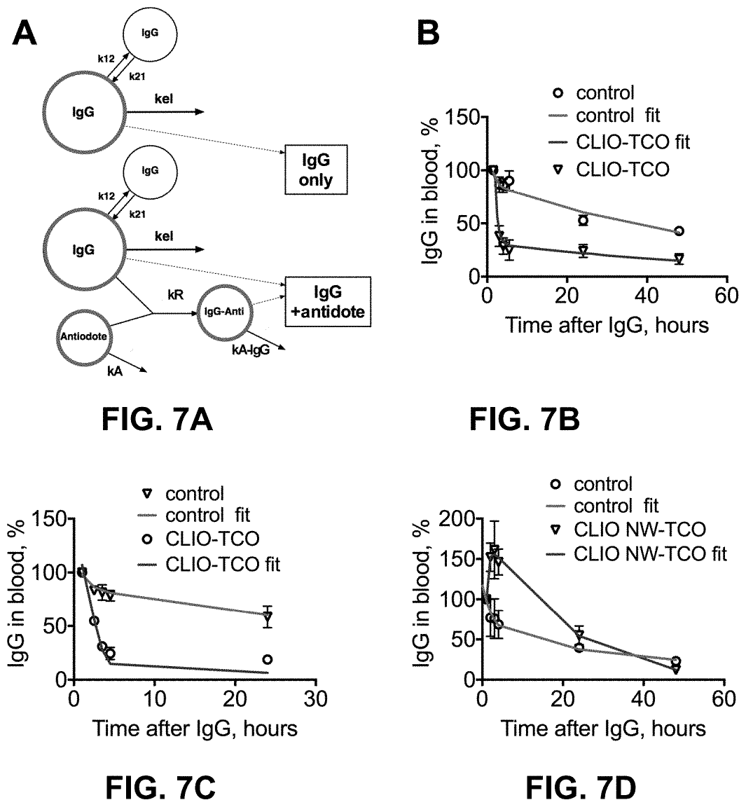

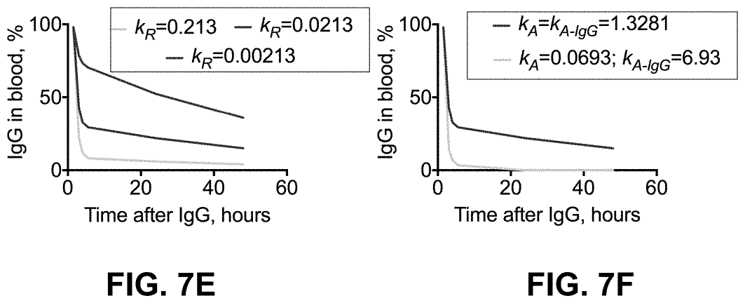

Pharmacokinetic modeling: IgG measurements versus time data without the antidote was modeled with a one- or two-compartment pharmacokinetic model using the pharmacokinetic modeling software Boomer. Each data point was weighted equally. The two-compartment model was found to provide a better fit to the IgG data after consideration of the AIC (Akaike Information Criterion) values, weighted residual plots and observed versus calculated concentration versus time plots. Data collected before and after administration of one or more doses of the antidote were added to the model and fitted simultaneously with a parallel model (FIG. 7A, top and bottom diagrams show the model without and with antidote, respectively), which included a second order interaction between IgG and the antidote and elimination of the antidote-IgG complex. In the presence of the antidote the IgG signal included both the free and bound IgG. In each case, a fit to both the free IgG and antidote-bound IgG resulted in random weighted residual plots and good correspondence between observed and calculated data versus time plots.

### EXAMPLES

### Example 1

### Click-Modified Nanocarriers

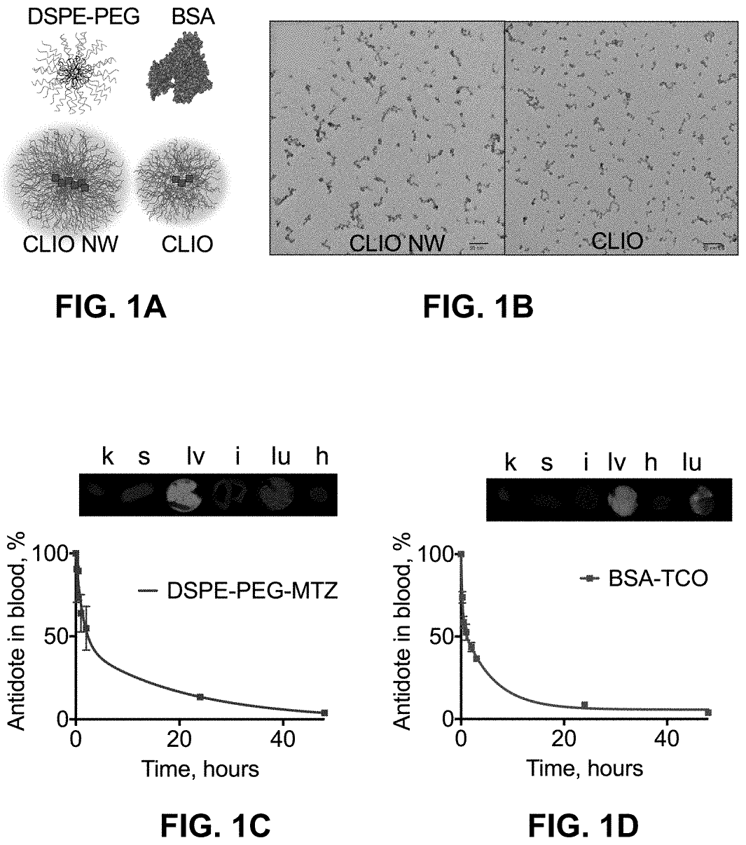

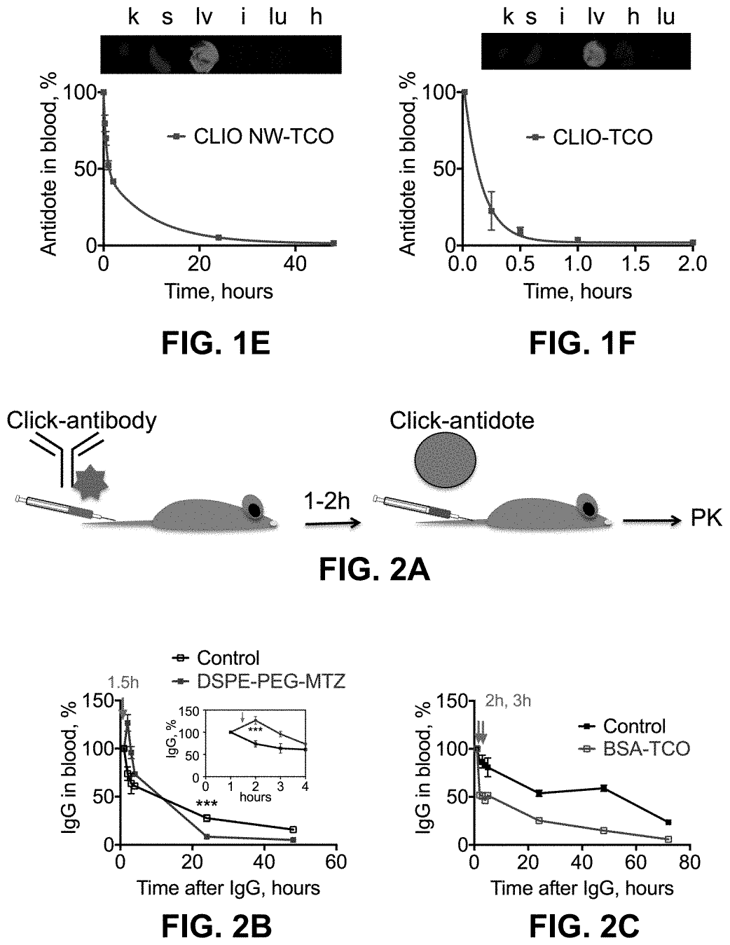

In order to develop antidotes that capture and eliminate antibodies in vivo, click-modified carriers of different chemistries and sizes were prepared (FIG. 1A). Phospholipid micelles are some of the most attractive delivery systems, and PEGylated phospholipids are FDA-approved components of liposomal drugs (e.g., Doxil®, Onivyde®). An MTZ derivative of DSPE-PEG3400 was prepared as described above. The lipid formed heterogeneous assemblies sized between 17 nm and 257 nm and a negative zeta potential of −6 mV due to the presence of the phosphate group. Theoretical binding capacity (based on weight fraction of MTZ groups) was 1.5 μg of IgG per μg lipid (Table 1). The actual binding capacity is likely to be lower, since the surface area of the lipid assemblies should limit the number of bound IgG molecules. To prepare a macromolecule-based antidote, we conjugated NHS-PEG4-TCO to bovine serum albumin (BSA). BSA-TCO was 8 nm in diameter with a zeta potential of −20 mV, and a theoretical binding capacity of 2 IgG/BSA or 2.3 μg IgG/μg BSA.

Superparamagnetic crystalline iron oxide nanoparticles (SPIO) are an important magnetic resonance imaging contrast agent that is also used as a component of multifunctional theranostic nanomedicines for imaging and treatment. To prepare nanoparticle-sized antidote, aminated cross-linked 20 k Da dextran CLIO nanoworms (CLIONW), and aminated cross-linked 10 k Da dextran CLIO nanoparticles (CLIO, FIG. 1A) were synthesized.

According to TEM images (FIG. 1B), CLIONWs contained mostly worm like cores composed of several Fe₃O₄ crystals, whereas CLIO had smaller size cores and there were many single crystalline nanoparticles. Each CLIO NW particle contained ˜30,000 amino groups and each CLIO particle contained ˜6,000 amino groups. These amines were further modified with NHS-PEG4-TCO in order to form CLIONW-TCO and CLIOTCO.

The hydrodynamic diameter of CLIONW-TCO and CLIO-TCO was 70 nm and 55 nm, and zeta potential was −2.7 mV and +8 mV, respectively. Theoretical binding capacity (based on the surface area) was 95 and 160 IgG/particle, or 1.9 and 2.3 μg IgG/μg Fe, respectively (Table 1). To measure the blood half-life of the antidotes, DSPE-PEG-MTZ micelles were labeled with DiR, while BSA-TCO, CLIONW-TCO and CLIO-TCO were labeled with IRDye 800CW. The elimination half-life following i.v. injection into female BALB/c mice was in the following order (slow phase): DSPE-PEG-MTZ (14 h) >CLIO NW-TCO (6.7 h) >BSA-TCO (4 h) >>CLIO-TCO (6 min) (FIGS. 1C-1F). The shorter half-life of CLIOTCO versus CLIONW-TCO was probably due to excess positive charge on the former, since positively charged particles are usually cleared faster than negatively charged ones. All the antidotes accumulated predominantly in the liver and spleen, with minor accumulation in the kidney (FIGS. 1C-1F). In addition, DSPE-PEG-MTZ and BSA-TCO showed significant accumulation in the lungs (FIGS. 1C, 1D).

TABLE 1
Measured and calculated parameters of the antidotes:

| | | | | ζ− | | Theoretical | Capacity, |
|---|---|---|---|---|---|---|---|
| | | Diameter, | | potential, | Particles | IgG/NP | μg IgG/ |
| Name | Type | nm | PDI | mV | per mg | (mole ratio) | μg NP |
| DSPE-PEG- | Lipid assembly | 17(43%); | N/A | −5.94 | N/A | N/A | ~42 |
| MTZ | | 257(57%) | | | | | |
| BSA-TCO | Macromolecule | 8 | N/A | −20.3 | 9 × 10¹² | ~2 | ~2.3 |
| CLIO NW- | Crosslinked | 70 | 0.3 | −2.7 | ~6 × 10¹⁰ | ~160 | ~2.3 |
| TCO | 20 kDa dextran | | | | | | |
| | iron oxide | | | | | | |
| CLIO-TCO | Crosslinked | 55 | 0.27 | 7.9 | ~8 × 10¹⁰ | ~95 | ~1.9 |
| | 10 kDa dextran | | | | | | |
| | iron oxide | | | | | | |

BSA-TCO size and zeta potential are similar to those of native BSA values reported in literature. Particle concentration of BSA was determined based on Mw of 67 kDa; CLIO and CLIONW concentration was determined as described before. Particle concentration of lipid assemblies could not be estimated due to size heterogeneity. Theoretical surface-based capacity (bound IgG/particle) was determined from particle surface area and assuming that each IgG molecule occupies a cross section of 100 nm². The binding capacity was calculated from surface based capacity, or in case of DSPE-PEG-MTZ based on percent weight of MTZ groups (Mw 3613 Da).

### Example 2

### In Vivo Efficiency of the Antidotes

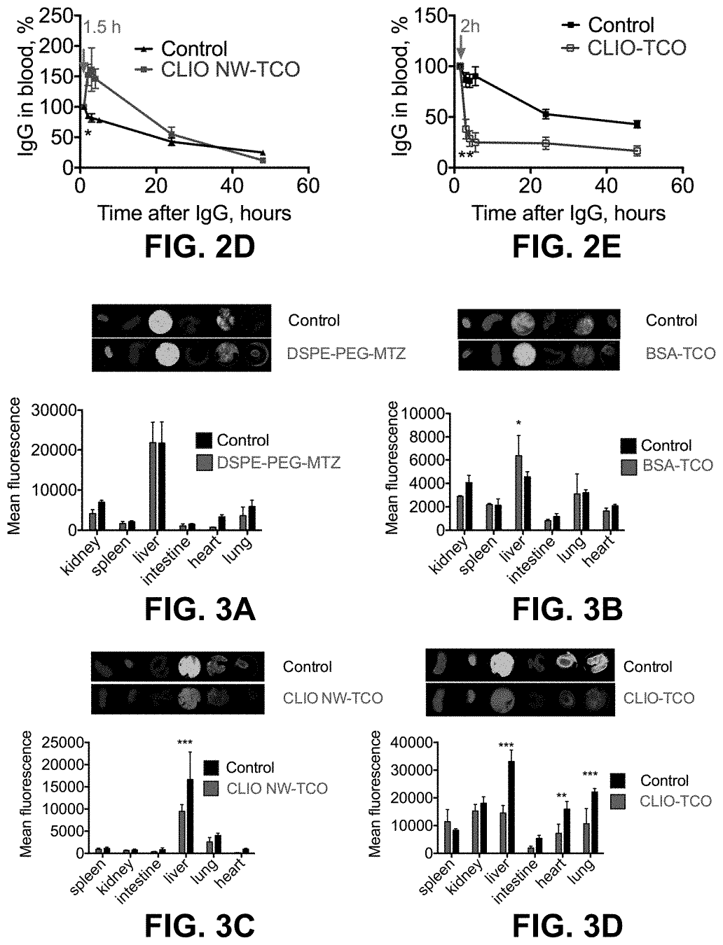

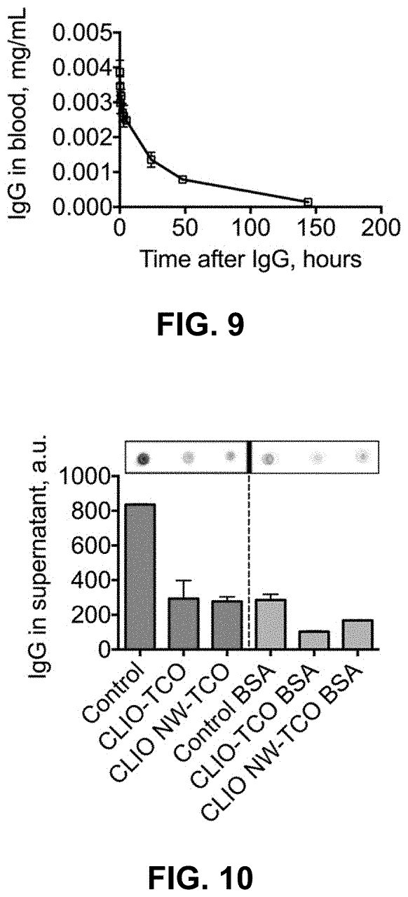

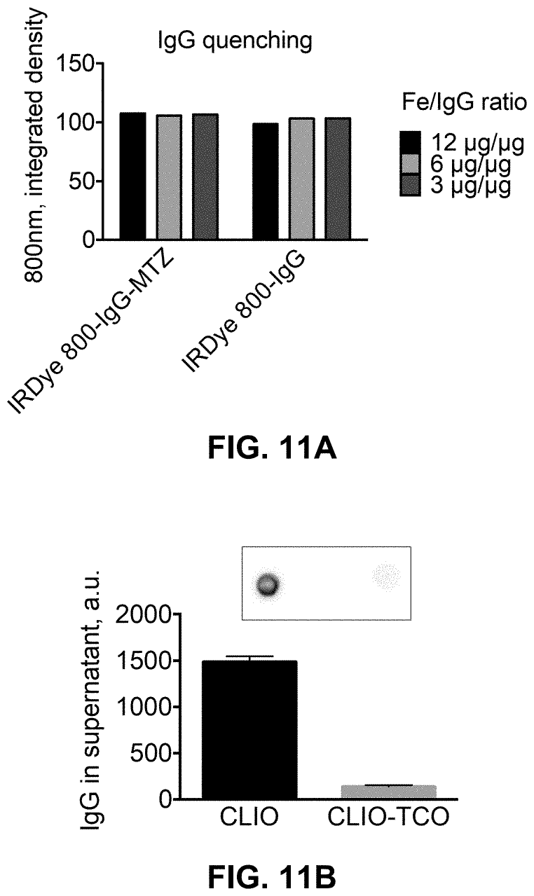

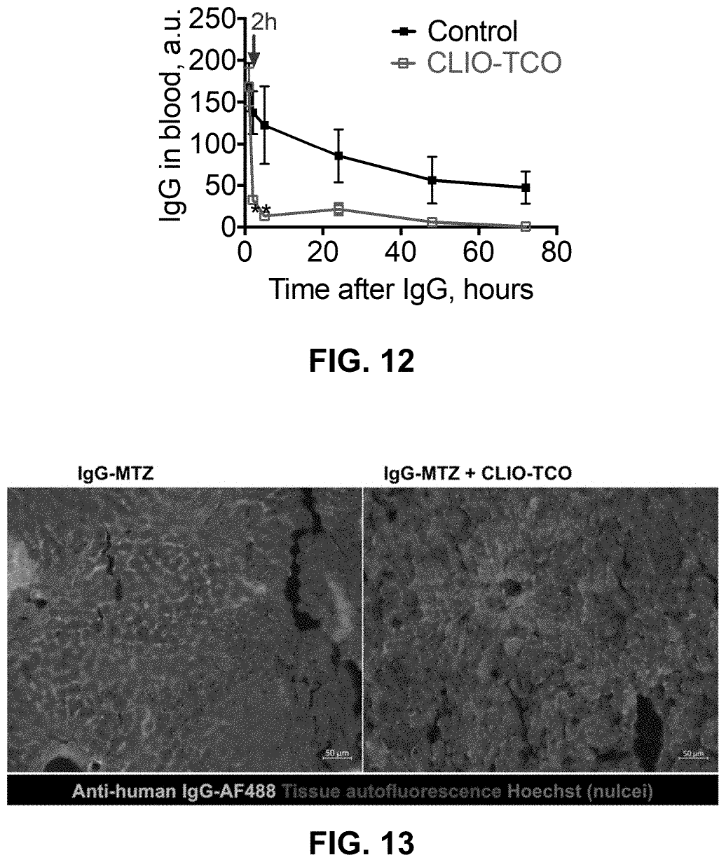

In order to test the depletion efficiency of the antidotes, female BALB/c mice were injected with 25 μg IRDye 800CW-labeled IgG followed by PBS (control) or the antidote, and the levels of IgG in blood (both antidote-bound and free) were measured at different times post-IgG injection (FIG. 2A). IgG alone has a long biexponential blood half-life of 32 h (slow phase, FIG. 9); however, in order to account for experimental variability and batch effect of the modified antibodies, each depletion experiment always included an “IgG only” group along with a “IgG+antidote” group. To test the ability of DSPE-PEG-MTZ to decrease total blood levels of IgG (both antidote bound and free), mice were injected with IRDye 800CW-IgG-TCO followed 1.5 h later with the lipid (1 mg, 1660-fold molar excess of MTZ groups). Surprisingly, there was a 72% increase in the blood levels of IgG at 2 h (p-value 0.0007, 2 sided t-test, n=3), and a 68% decrease at 24 h (p-value 0.00018, 2 sided t-test, n=3) post IgG injection, compared to the control IgG group (FIG. 2B). Overall, however, the injection of DSPE-PEG-MTZ did not significantly decrease the AUC24 h (17.7%; p-value 0.056, 2-tailed t-test, n=3). To test the depletion efficiency of BSA-TCO, we injected mice with 25 μg of IgG-MTZ followed by BSA-TCO at 2 h and 3 h post-IgG injection (500 μg each; ˜400 fold excess of TCO; 92-fold excess of surface-binding capacity). BSA-TCO decreased the blood level of IgG by 40% at 3 h (FIG. 2C; p-value <0.0001, 2 sided t-test, n=3) and by 53% at 24 h, and the AUC24 h was significantly decreased (41%; p-value <0.0001, 2-tailed t-test, n=3) compared to the control group. In view of the large excess of the injected lipid and BSA over IgG, it is unlikely that insufficient binding capacity is responsible for the observed difference in the depletion efficiency of these antidotes. To further investigate which factors contribute to the depletion efficiency, CLIO-TCO and CLIO NW-TCO were compared that have similar sizes but different blood half-lives (FIGS. 2E, 2F). Both nanoparticles showed similar IgG-MTZ binding capacity in BSA and mouse blood (FIG. 10). 25 μg of IRDye 800CW-IgG-MTZ was injected followed 1.5 h later with long-circulating CLIO NWTCO (290 μg Fe; ˜5200-fold molar excess of TCO, 26.5-fold excess of surface-binding capacity). The injection resulted in a 68% increase in the blood level of IgG at 2 h post-IgG injection (FIG. 2D; p-value 0.01, 2 sided t-test, n=3) and non-significant increase at 24 h, compared to the control group (FIG. 2D; p-value 0.12, 2 sided t-test, n=3). The AUC24 was significantly increased (85.6%; p-value<0.0001, 2-tailed t-test, n=3). On the other hand, injection of short circulating CLIO-TCO 1 h after IgG (290 μg; ˜1400-fold molar excess of TCO over IgG, 22-fold excess of surface-binding capacity) resulted in 56% decrease in IgG 3 h post-IgG injection (p-value 0.0022, 2 sided t-test, n=3), 54% decrease at 24 h, and significant decrease in AUC24 h (62%; p-value<0.0001, 2-tailed t-test, n=3) compared to the control IgG group (FIG. 2E). Conjugation of IgG-MTZ to CLIO-TCO did not result in any decrease in the fluorescence of the antibody (FIG. 11A). It was also confirmed that CLIO-TCO specifically binds to IgG via click chemistry (FIG. 11B), which confirms that the observed decrease in IgG blood fluorescence was due to depletion. Furthermore, to exclude the possibility that near infrared dye IRDye800 detached from the antibody in the circulation, we confirmed the similar efficiency of depletion of IgG-MTZ by CLIO-TCO using secondary antihuman antibody (FIG. 12).

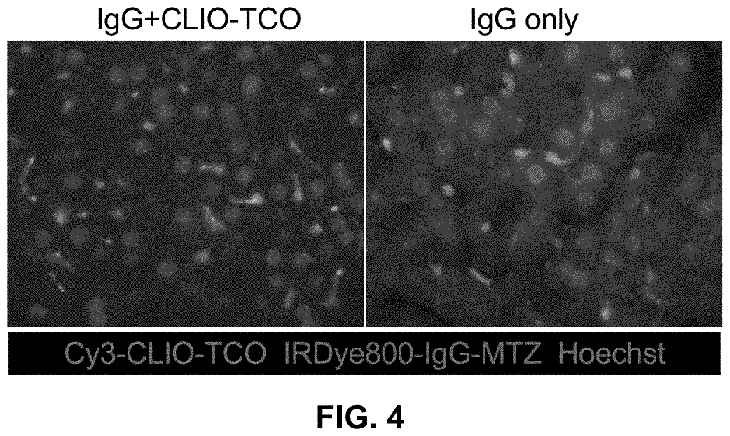

To study the depletion in the peripheral organs, the main organs were scanned 48-72 h after the antidote injection with Li-COR Odyssey NIR scanner. The mean fluorescence reflects both the deposited IgG and the circulating IgG (either free or antidote-bound). According to FIGS. 3A and 3C, DSPE-PEG-MTZ and CLIO NW-TCO did not cause any decrease in the fluorescence in the organs. BSA-TCO (FIG. 3B) actually increased the liver fluorescence (p-value 0.02, 2-way ANOVA with multiple comparisons, n=3) but did not decrease the level of fluorescence in other organs. CLIO-TCO (FIG. 3D) that had the best depletion efficiency in blood also decreased fluorescence by 50% in the liver and the lungs (liver p-value <0.0001; lung p-value <0.001; 2-way ANOVA with multiple comparisons, n=3). The decreased fluorescence in the organs probably reflected the decrease in the blood pool levels of IgG (the antibody was still in circulation when the animals were euthanized). Liver is the main clearance organ for CLIO-TCO (FIG. 9). In order to understand how the antidote injection affects distribution of IgG in the liver, we injected mice with IRDye800-IgG-MTZ alone or followed by Cy3-CLIO-TCO, euthanized 3 h post-antidote injection and imaged the histological sections with fluorescence microscope. As shown in FIG. 4A, antidote injected mouse showed co-localization of the nanoparticles and the antibody in the Kupffer cells, whereas control IgG-injected mouse showed diffuse and widespread distribution consistent with the antibody being in the blood pool and also in hepatocytes and endothelial cells (FIG. 4B). A similar result was obtained by indirect immunofluorescence using a secondary anti-human antibody to detect human IgG in the liver (FIG. 13). These data suggest that the antidote mainly directs the antibody to the Kupffer cells in the liver.

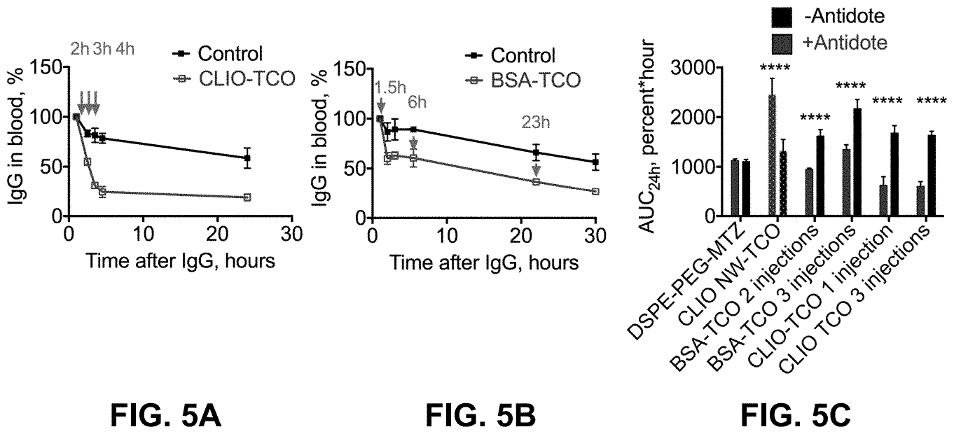

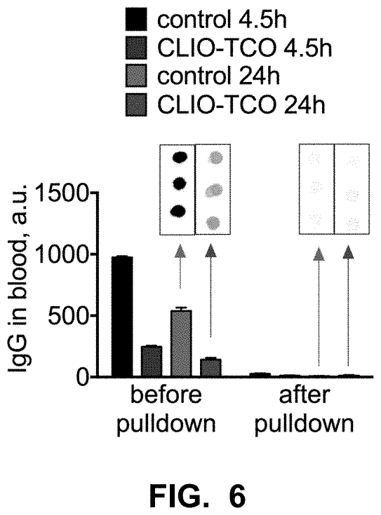

Two injections of BSA-TCO and a single injection of CLIO-TCO showed efficient depletion, but there was still a substantial fraction of non-eliminated IgG in blood (FIGS. 2C, 2E). Injection of mice with 25 μg IRDye 800CW-IgG-MTZ followed with 3 injections of BSA-TCO at 1.5 h, 3 h and 23 h post-IgG (500 μg each; ˜1200-fold excess TCO, ˜140-fold excess of surface-binding capacity) showed a 38% decrease in AUC24 h (p value <0.0001, 2-tailed t-test, n=3), which was not different from that achieved after 2 injections of BSA-TCO (FIGS. 5A, 5C). Three injections of CLIO-TCO at 2 h, 3 h and 4 h post-IgG (150 μg each; ˜2300 fold excess TCO, 34-fold excess of surface-binding capacity) showed 62.7% decrease in AUC24 h (p-value<0.0001, 2-tailed t-test, n=3), which was not different from that achieved after a single injection of 290 μg of CLIO-TCO (FIG. 5B, FIG. 5C). These data suggest that multiple injections of the antidote are not able to clear the remaining circulating IgG. To exclude the possibility that the remaining fraction of IgG does not have the reactive MTZ group, blood samples were drawn from CLIO-TCO injected mice and control mice at 4.5 h and 24 h (FIG. 2E) and added excess of CLIO-TCO. The particles were pelleted with ultracentrifugation and the supernatant NIR fluorescence was measured with Li-COR Odyssey. According to FIG. 6, IgG was completely pulled down by the nanoparticles at both time points, ruling out the possibility that the remaining circulating IgG was not reactive.

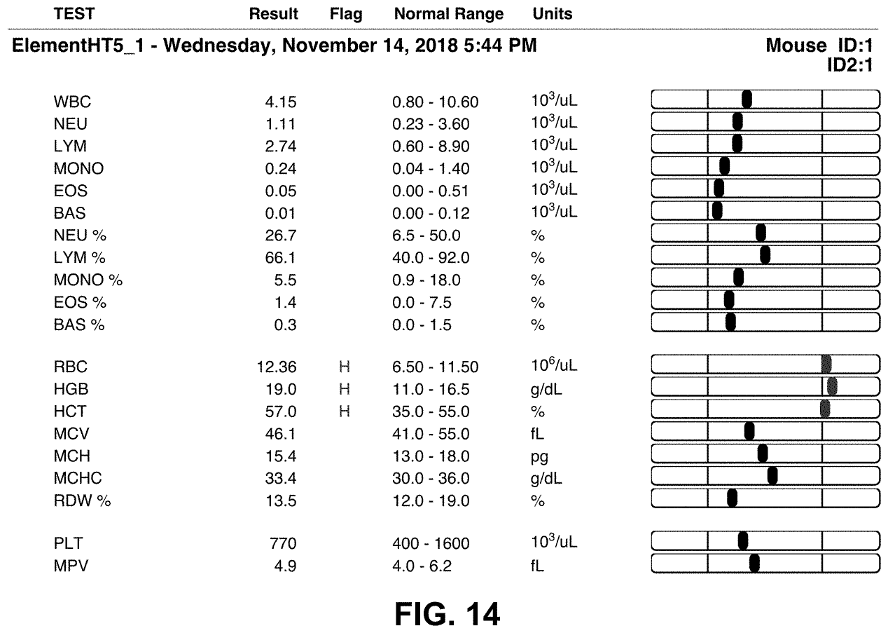

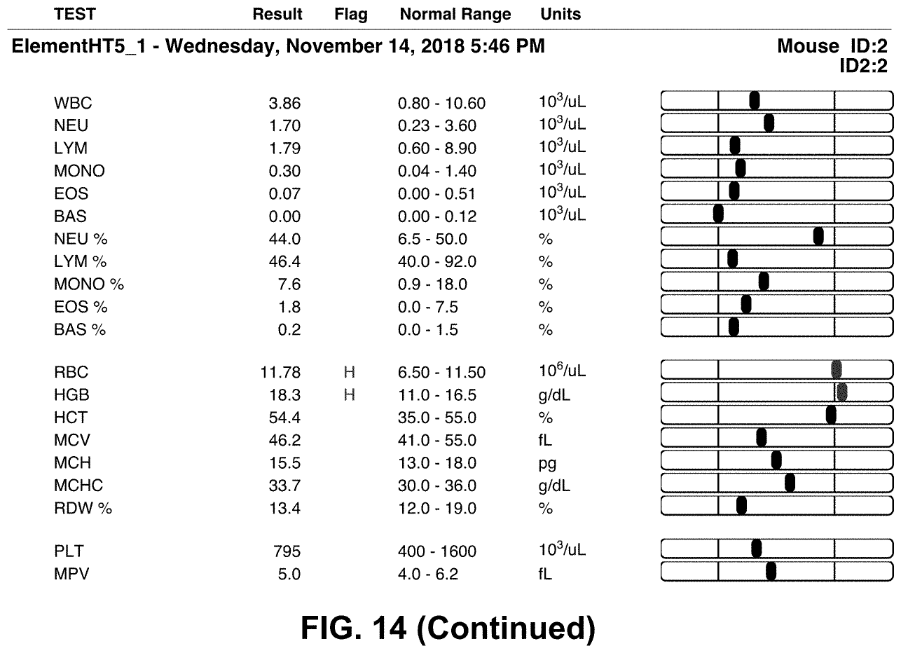

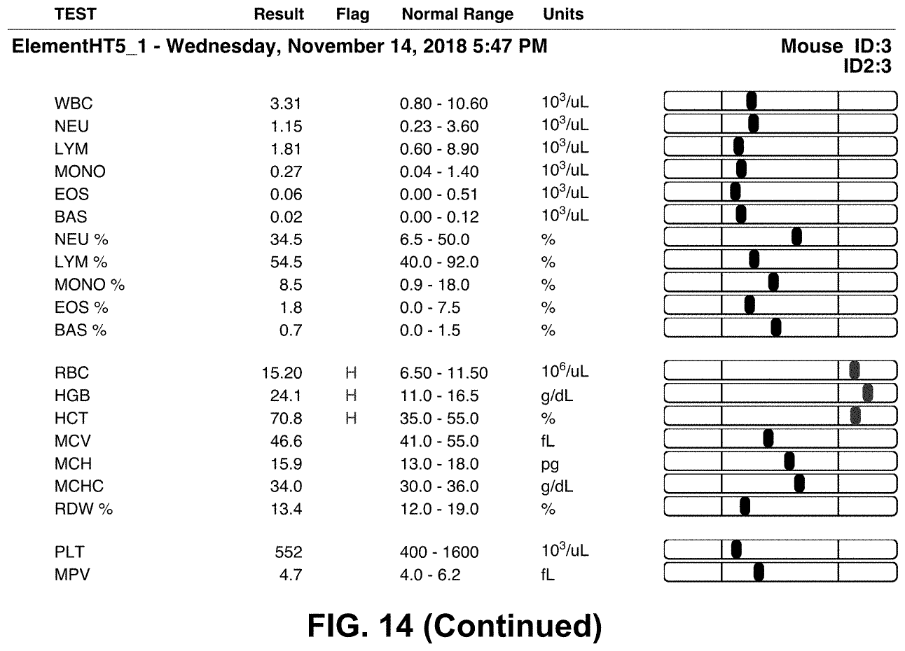

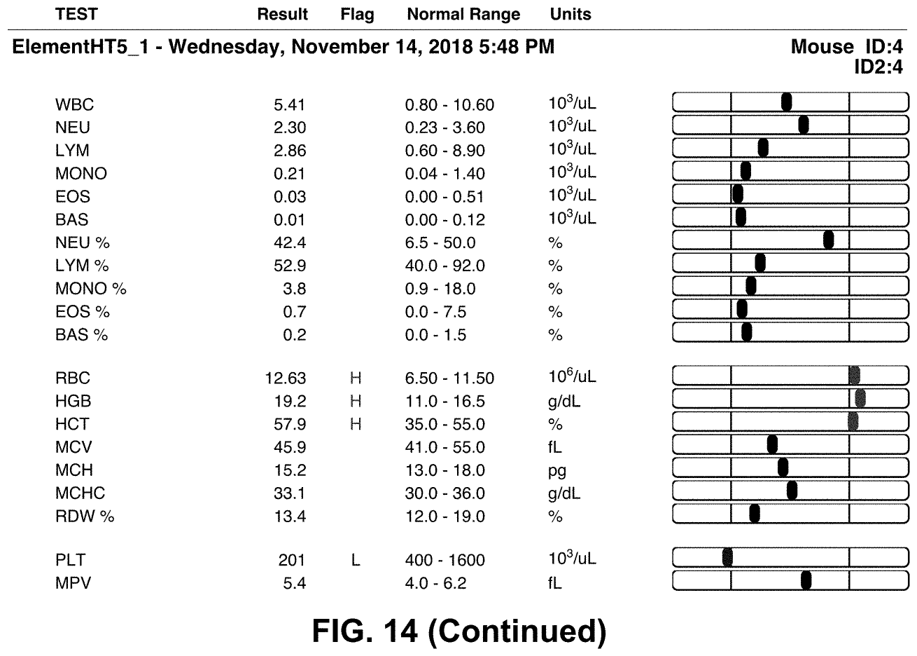

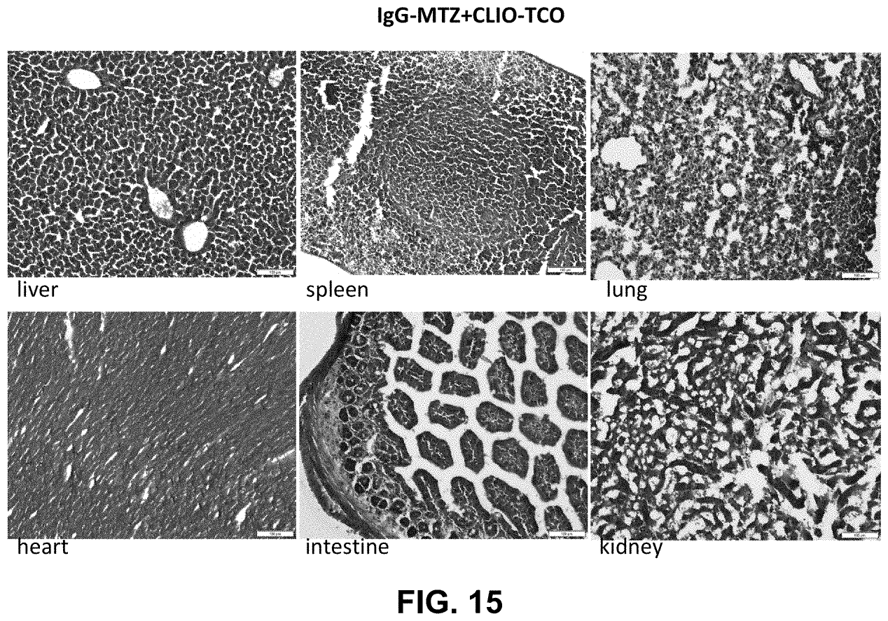

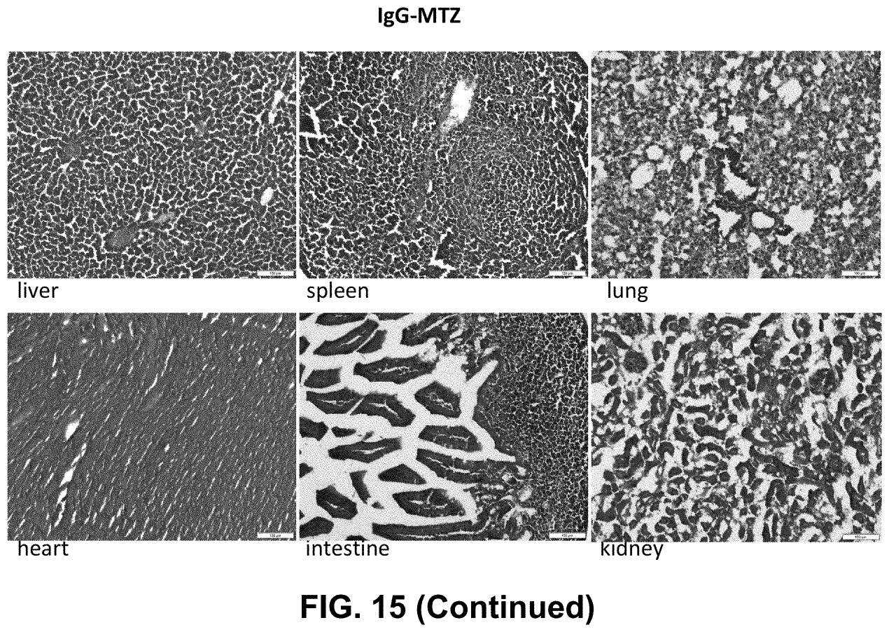

CLIO-TCO nanoparticles were further assessed to determine whether they induce toxicity. Nanoparticle toxicity often causes challenges in clinical development of nanomaterials. Analysis of blood cell profile of mice injected with IgG-MTZ followed by CLIO-TCO (4 h post-IgG injection, FIG. 14) and hematoxylin-eosin staining of the main organs (FIG. 15) did not reveal any gross differences with IgG-MTZ injected mice and no signs of inflammatory response.

### Example 3

### Modeling of Parameters that Determine Depletion Efficiency

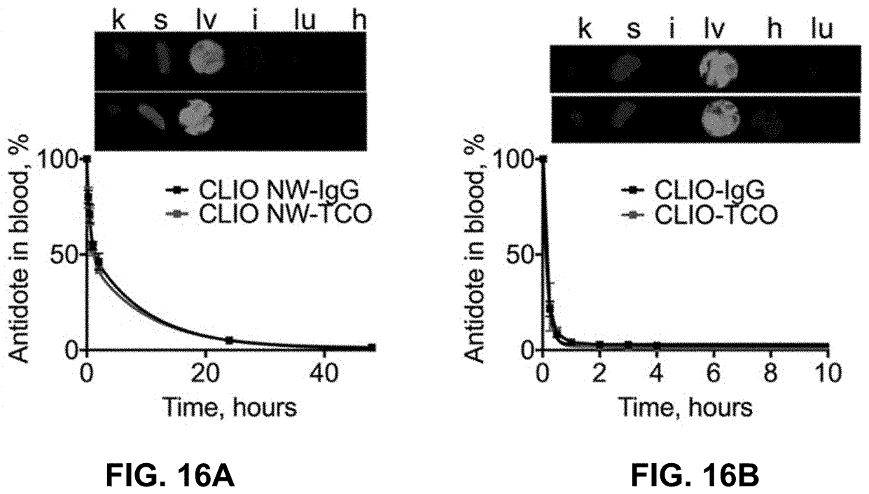

CLIO and CLIO NWs have similar size, IgG binding efficiency and biodistribution, but different half-lives and strikingly different depletion efficiency. In order to understand the factors that determine the depletion efficiency, we developed a compartmental pharmacokinetic (PK) model that takes into account blood elimination rates of the antidote and the antidote-IgG complex, click reaction rates and the antibody exchange rate between blood and tissue compartment (FIG. 7A). Using our previously described PK modeling program (Boomer 34) we calculated the volume of distribution V and tissue/blood exchange rates k₁₂ and k₂₁ of IgG only (Table 2 and FIG. 9). These parameters were adjusted for each experiment by the software in order to obtain the best fit to the antidote data. The elimination rate of CLIO-TCO and CLIONW-TCO conjugated to IgG-MTZ was the same as of free antidotes (FIGS. 16A-16B). The model produced an accurate fit for IgG blood profile (FIGS. 7B-7C) after injection of CLIOTCO (1 injection) (Table 2 for the fitted parameters) and a reasonable fit for CLIO-TCO (3 injections). The model predicts that multiple injections of CLIO-TCO do not improve depletion of blood IgG at early time points (FIG. 7C), but predicts a slightly better depletion at 24 h than observed experimentally. The mechanism of incomplete IgG depletion despite click reactivity of the remaining antibody (FIG. 6) is likely due to the exchange of the IgG between blood and tissue compartments, which may not be fully accounted for by the PK model. The model produced an excellent fit for long-circulating CLIONW-TCO and predicted the observed spike in IgG at 2 h and decrease at 24 h (FIG. 7D). Interestingly, the simulation also explains the result obtained with DSPE-PEGMTZ, which had a long circulation time and showed the spike in IgG levels and depletion at late time points. Simulations of click reaction rate constant showed strong effect on the depletion efficiency (FIG. 7E). Click rates in vitro reach up to 3.8×10⁵ M⁻¹ s⁻¹, but the rate in vivo could be lower and depend on the interaction between antidote, blood cells and blood proteins. Finally, IgG depletion efficiency was simulated by a hypothetical antidote that has a long blood half-life but exhibits accelerated clearance upon click reaction with IgG (FIG. 7F). The modeling predicts that such an antidote should exhibit the best efficiency due to long residence in blood and quick removal of the IgG that diffuses from the tissue back into blood compartment.

TABLE 2
Parameters of the model

| Experiment/ | IgG/CLIO-TCO | IgG/CLIO NWTCO | IgG only |
|---|---|---|---|
| Parameter | (FIGS. 2E, 5B) | (FIG. 2D) | (FIG. 9) |
| kel (1, 2) | 0.0264 | 0.0345 | 0.031 |
| k12 | 0.426 | 0.16 | 0.106 |
| k21 | 0.655 | 0.188 | 0.181 |
| VIgG | 0.168 | 0.213 | 7.068 ml |
| VIgG-Antidote | 0.62 | 0.233 | N/A |
| kA; kA-IgG | 1.33/1.174 | 0.0632/0.087 | N/A |
| kR | 0.0213 | 0.0168 | N/A |

The parameter labels correspond to the scheme in FIG. 7A. Parameters such as apparent volume of distribution V in the antidote experiments are unitless due to fitting to the percent IgG over time profile. All k rate constants are h⁻¹. The program (Boomer) was allowed to adjust the parameters to obtain the best fit. Note that the model-adjusted parameters are close to, but not the same as, the parameters obtained from fitting actual data (bold). Elimination rate constants of antidotes and IgG-antidote complexes is assumed to be similar and monoexponential (for the sake of simplicity of the model). IgG disposition was found to be biexponential due to distribution into tissue compartment.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Whereas certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A pharmaceutical composition for reducing antibodies combined with either trans-cycloctene (TCO) and methyltetrazine (MTZ) comprising a copper-free click chemistry component and a nanoparticle drug carrier.

2. A composition of claim 1 wherein the copper-free click chemistry component is selected from the group consisting of trans-cycloctene (TCO) and methyltetrazine (MTZ).

3. A composition of claim 1 wherein the nanoparticle drug carrier is selected from the group consisting of phospholipid-PEG micelles, bovine serum albumin (BSA), cross-linked dextran iron oxide nanoparticles (CLIO) and cross-linked dextran iron oxide nanoparticle nanoworms (CLIO-NW).

4. The composition of claim 3 wherein the nanoparticle drug carrier is cross-linked dextran iron oxide nanoparticles (CLIO).

5. The composition of claim 3 wherein the nanoparticle drug carrier is cross-linked dextran iron oxide nanoparticle nanoworms (CLIO-NW).

6. The composition of claim 4 wherein the copper-free click chemistry component is trans-cycloctene (TCO).

7. A pharmaceutical composition comprising a compound of claim 1 and at least one pharmaceutically acceptable additive.

8. A pharmaceutical kit containing a pharmaceutical compound of claim 7, prescribing information for the composition, and a container.

9. A method of reducing or eliminating antibodies combined with either trans-cycloctene (TCO) and methyltetrazine (MTZ) from a body comprising administering to a subject a therapeutically-effective amount of a copper-free click chemistry modified nanoparticle composition.

10. The method of claim 9 wherein the nanoparticle composition is modified with a copper-free click chemistry component selected from the group consisting of trans-cycloctene (TCO) and methyltetrazine (MTZ).

11. The method of claim 9 wherein the nanoparticle composition is selected from the group consisting of phospholipid-PEG micelles, bovine serum albumin (BSA), and cross-linked dextran iron oxide nanoparticles (CLIO).

12. The method of claim 9 wherein a concentration of said antibodies is reduced by at least 50%.
